(19) United States
(12) Patent Application Publication
(10) Pub. No.: US 2008/0206782 A1
(43) Pub. Date: Aug. 28, 2008

(54) Methods for Determining the Bivalency of Protein and Antibody Therapeutics

(75) Inventors: Shani Gunasena Golden, Houston, TX (US); Mangyuan Patrick Liu, Pearland, TX (US)

Correspondence Address: Nikolaos C. George;JONES DAY, 222 E. 41st. Street, New York, NY, 10017-6702, US

(73) Assignee: Genentech, Inc., South San Francisco, CA (US)

(21) Appl. No.: 11/885,917

(22) PCT Filed: Mar. 7, 2006
(86) PCT No.: PCT/US06/08041

§ 371 Date: Sep. 7, 2007

Related U.S. Application Data
(60) Provisional application No. 60/659,547, filed on Mar. 8, 2005.

(51) Int. Cl. G01N 33/53; G01N 33/566

(52) U.S. Cl. 435/7.1; 436/501

(57) ABSTRACT

The invention provides methods and kits for detecting or quantitating intact bivalent antibody molecules in a sample and distinguishing those molecules from monovalent fragments.

### BACKGROUND OF THE INVENTION

Antibody therapeutics can be useful for treating a wide range of diseases including cancer, asthma, allergy, psoriasis, arthritis, cardiovascular diseases, autoimmune diseases such as Crohn's disease and Multiple Sclerosis, transplant rejection, and viral infection. The therapeutic potential of antibodies was first recognized in the early 1980's when mouse monoclonal antibodies were administered to humans for in vivo therapy. The therapeutic use of mouse monoclonal antibodies in humans, however, was limited due to their inability to trigger human effector functions coupled with a short serum half-life and the production of human anti-mouse antibodies (for a review see, Brekke and Sandlie, (2003) Nature Reviews 2:52-62). To overcome the inability of mouse monoclonal antibodies to trigger human effector functions and to reduce immunogenicity, antibodies were engineered to generate chimeric and humanized antibodies. Antibody fragments were also designed for clinical applications (for a review see, Hudson and Souriau, (2003) Nature Medicine 9:129-134). For example, antibody fragments including Fabs and scFvs can more effectively penetrate target tissues compared to full intact antibodies, and thus, in some cases are more effective in delivering toxins to a tumor site. Today, monoclonal antibodies and antibody fragments are genetically engineered for high specificity, and functionality for use in a variety of clinical applications. Immunoglobulin IgG4 subclass tend to loose the bivalency in vivo due to instable disulfide bond between the two heavy chains through half molecular exchange with other IgG4 molecules. IgG4 molecules in some circumstances must be in their bivalent form to be biologically effective. Therefore, for these therapeutic antibodies of IgG4 it is important to quantitate the amount of the therapeutics present in their bivalent form.

The clinical effectiveness of antibody drugs is determined through clinical studies. For example, such studies evaluate the accessibility of the antibody drug to its target, half-life in serum, and the ability to effect biological functions of its target. Antibodies are also characterized for their physical properties (e.g., size of the intact antibody or antibody fragment) using techniques such as gel electrophoresis and size-exclusion chromatography. Bivalency of an antibody can be determined by biosensors (Conrath et al., (2001) J. Biol. Chem. 276:7346-7350). These methods of characterizing antibodies, however, are not applicable for efficiently analyzing the amount of therapeutic antibody that may be in a patient sample at any given time. Further, methods that detect antibodies based on binding epitopes cannot distinguish an intact, functional antibody from a degraded antibody fragment.

Rapid, sensitive, and high-throughput methods are needed to analyze patient samples for functional, intact antibody drugs.

### SUMMARY OF THE INVENTION

In one aspect, the present invention provides methods for detecting or quantitating antibodies in a sample by incubating the sample with capture anti-idiotypic antibodies or target molecule (“capture agent”) and detection anti-idiotypic antibodies or target molecule (“detection agent”) under appropriate conditions and for a sufficient period of time to allow binding, wherein detection of bound immune complex is directly proportional to the relative amount of antibodies present in the sample. In some cases either capture or detection agent can be the target molecule of the therapeutic antibody, or in other words the target molecule for which the therapeutic antibody is supposed to bind. In some other cases, both capture and detection agent can be the same target molecule defined above. In certain embodiments the capture agent is linked (immobilized) to a solid support. In other embodiments the detection agent includes secondary detection system, such as secondary antibodies, biotin-avidin system.

In a second aspect, the invention provides kits for detecting or quantitating a antibody in a sample, comprising: a capture agent binding to the antibody and a detection agent binding to antibody. The kit may further comprise a solid support.

The instant described methods and kits provide a rapid, sensitive and high-throughput means for analyzing patient samples for intact antibodies and can distinguish or screen out monovalent or split molecules from intact functional antibodies. The disclosed methods and kits can therefore be useful for monitoring the effectiveness of antibody therapeutics. Other features and advantages of the invention will be readily apparent to the skilled artisan based on the following Detailed Description and Claims.

### BRIEF DESCRIPTION OF THE FIGURE

FIG. 1 is a schematic diagram of an assay format illustrating the capture agent coated well of a microtiter plate and a complex formed with the test antibody, which also binds to a detection agent comprising a detectable label.

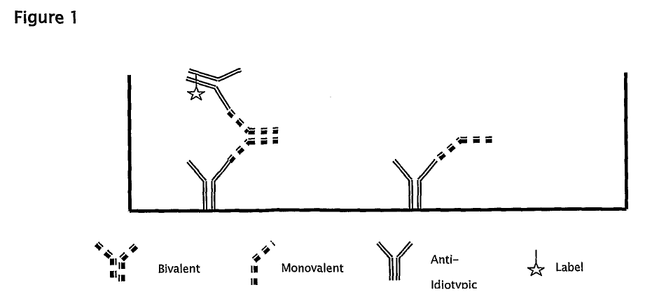

### DETAILED DESCRIPTION OF INVENTION

This invention is not limited to the particular a methodology, protocol, or reagents described herein because they may vary. Further, the terminology used herein is for the purpose of describing particular embodiments only and is not intended to limit the scope of the present invention. As used herein and in the appended claims, the singular forms “a”, “an”, and “the” include plural reference unless the context clearly dictates otherwise, e.g., reference to “an antibody cell” includes a plurality of such antibodies.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

All patents and publications mentioned herein are incorporated herein by reference to the extent allowed by law for the purpose of describing and disclosing the proteins, enzymes, vectors, host cells, and methodologies reported therein that might be used with the present invention. However, nothing herein is to be construed as an admission that the invention is not entitled to antedate such disclosure by virtue of prior invention.

### Definitions

For convenience, certain terms employed in the specification, examples, and appended claims are provided below.

An “antigen binding site” as used herein, refers to the variable domain of a heavy chain associated with the variable domain of a light chain.

An “anti-idiotypic antibody” or “anti-id,” as used herein, refers to a molecule that contains an antigen-binding site specific for at least a portion of the variable region of a test antibody. For example, anti-idiotypic antibodies may be raised as polyclonal or monoclonal antibodies from animals immunized with a test antibody.

“Body fluid,” as used herein, refers to a fluid that is obtained from a subject or is further processed from a fluid obtained from a subject. Examples include: blood, plasma, urine, interstitial fluid, lymph, gastric juices, bile, serum, saliva, sweat, spinal fluids and brain fluids.

The term “capture agent” as used herein, refers to an anti-idiotypic antibody or target molecule on a solid support through a direct or indirect linkage, chemically or immunobiologically.

“Reference standards” refer to known quantities of test antibody that have been quantitated and added to or spiked in a sample. Reference standards may be used to generate a standard curve.

The term “detection agent” as used herein, refers to an anti-idiotypic antibody or target molecule with detectable label, or linked to a secondary detection system

“Fluorophore” refers to a substance or a portion thereof which is capable of exhibiting fluorescence in the detectable range. Fluorophores that may be conjugated to a primary antibody include, but are not limited to, Fluorescein, Rhodamine, Texas Red, Cy2, Cy3, Cy5, VECTOR™ Red, ELF™ (Enzyme-Labeled Fluorescence), Cy0, Cy0.5, Cy1, Cy1.5, Cy3, Cy3.5, Cy5, Cy7, FluorX, Calcein, Calcein-AM, CRYPTOFLUOR'S™, Orange (42 kDa), Tangerine (35 kDa), Gold (31 kDa), Red (42 kDa), Crimson (40 kDa), BHMP, BHDMAP, Br-Oregon, Lucifer Yellow, Alexa dye family, N-[6-(7-nitrobenz-2-oxa-1,3-diazol-4-yl)amino]caproyl] (NBD), BODIPY™, boron dipyrromethene difluoride, Oregon Green, MITOTRACKER™ Red, DiOC₇ (3), DiIC₁₈, Phycoerythri n, Phycooycanin, Allophycocyanin, o-phthaldehyde, Phycobiliproteins BPE (240 kDa) RPE (240 kDa) CPC (264 kDa) APC (104 kDa), Spectrum Blue, Spectrum Aqua, Spectrum Green, Spectrum Gold, Spectrum Orange, Spectrum Red, NADH, NADPH, FAD, Infra-Red (IR) Dyes, Cyclic GDP-Ribose (cGDPR), Calcofluor White, Lissamine, Umbelliferone, Tyrosine and Tryptophan. A wide variety of other fluorescent probes are available from and/or extensively described in the Handbook of Fluorescent Probes and Research Products 8^{th} Ed. (2001), available from Molecular Probes (Eugene, Oreg.), as well as many other manufacturers.

The term “immunoassay” refers to any assay that utilizes an antibody to specifically bind a target protein. Examples of immunoassays include, but are not limited to, immunblot assays, enzyme linked immunosorbent assay (“ELISA”), enzyme immunoassay (EIA). In an ELISA or EIA assay, a label enzyme and substrate are used to produce an amplified signal.

“Label” and “detectable label” refers to a detectable compound or composition, which can be conjugated directly or indirectly to a molecule or protein, e.g., an antibody. The label itself may be detectable (e.g., radioisotope labels or fluorescent labels) or, in the case of an enzymatic label, may catalyze a chemical alteration of a substrate compound or composition, which is detectable. A label may include, but is not limited to radioactive isotopes, fluorophores, chemiluminescent moieties, enzymes, enzyme substrates, enzyme cofactors, enzyme inhibitors, dyes, metal ions, ligands (e.g., biotin or haptens) and the like. Enzymes which can be used to detectably label the antibody include, but are not limited to, horseradish peroxidase, alkaline phosphatase, beta-galactosidase, glucose oxidase, malate dehydrogenase, staphylococcal nuclease, delta-5-steroid isomerase, yeast alcohol dehydrogenase, alpha-glycerophosphate, dehydrogenase, triose phosphate isomerase, asparaginase, ribonuclease, urease, catalase, glucose-6-phosphate dehydrogenase, glucoamylase and acetylcholinesterase.

The enzyme can also be directed at catalyzing a luminescence reaction of a substrate, such as, but not limited to, luciferase and aequorin, having a substantially non-soluble reaction product capable of luminescencing or of directing a second reaction of a second substrate, such as but not limited to, luciferine and ATP or coelenterazine and Ca++, having a luminescencing product.

A “monovalent antibody” as used herein, refers to any molecule that contains only one antigen-binding site, including, but not limited to Fab fragment, half molecule antibody (HL), bispecific antibody and antibody (H₂L₂) with one binding site occupied by neutralizing factors. Monovalent antibodies may be produced by fragmentation (e.g., chemical or enzymatic) of intact antibodies, recombinantly from a gene encoding half antibody sequences, or wholly or partially synthesized. The term “Fab” refers to an antibody fragment that is essentially equivalent to that obtained by digestion of the immunoglobulin (typically IgG) with the enzyme papain. The half molecule antibody refer to molecule consisting of one heavy chain and light chain covalently connected to one another by a polypeptide linker, which may be produced in vitro by using fragmentation or recombinant techniques, or in vivo through a biological process. The bispecific antibody refers to antibody molecule with two distinctive antigen binding sites which can not cross-link the same antigen. Bispecific antibody may be produced in vitro by using biochemical or recombinant techniques, or in vivo through a biological process. The neutralizing factors refer to any molecule which can binding to the antibody and occupied the target molecule binding site on the antibody and block the binding of the target molecule.

A “bivalent antibody,” as used herein, refers to antibody molecule that contains at least two antigen-binding sites which can cross-link the same antigen, including native antibodies or immunoglobulin molecules (e.g., IgG, IgE, IgM, IgD, and IgA) or subclass. These antibodies include, but are not limited to, polyclonal, monoclonal, chimeric antibodies, partially or fully humanized antibodies, (i.e., generated in a transgenic mouse expressing human immunoglobulin genes), camelized antibodies, F(ab′)₂

The term “sample,” as used herein, refers to biological material that contains antibodies. Biological material may be obtained from a subject and may include, for example, tissue, cells, or body fluid. In exemplary embodiments, the sample is a body fluid.

The terms “solid support” refers to any support that is capable of binding an antigen, antibody, or another molecule of the present invention. Well-known supports include glass (e.g., controlled pore glass), polysaccharides (e.g., agarose), polyacrylamides, polystyrene, polypropylene, polyethylene, dextran, nylon, amylases, natural and modified celluloses, gabbros, magnetite, polyvinyl alcohol, and silicones. The support material can have virtually any possible structural configuration so long as the coupled molecule is capable of binding to an antigen or antibody. Thus, the support configuration can be spherical, as in a bead, or cylindrical, as in the inside of surface of a test tube, or the external surface of a rod. Alternatively, the surface can be flat, such as a sheet, test strip or well of a microtiter assay plate. In exemplary embodiments, a solid phase is the well of a microtiter assay plate. In alternate embodiments, a solid phase is a cellulose or nylon membranes. In other embodiments, a solid phase may comprise a purification column (e.g., an affinity chromatography column).

A “subject” refers to a human or a non-human animal.

The term “target molecule” refers to the molecule to which the therapeutic antibody can bind and may be a target for treatment of a disease. The target molecule may play a critical role on the pathogenesis or mechanism of the disease.

### Assay

Sensitive and specific assays for detecting or quantitating bivalent target antibodies in a sample are described. In general, the present methods involve incubating the sample with a capture agent and a detection agent, under appropriate conditions and for a sufficient period of time to allow binding, wherein detection of bound immune complex is directly proportional to the relative amount of antibodies present in the sample.

The assays described herein use anti-idiotypic antibodies or target molecule both to capture and detect the bivalent antibody. In an exemplary embodiment, bivalent antibodies will be sandwiched between the capture agent bound to a solid support and detection agent, labeled with a detectable marker, in the liquid phase. Only bivalent antibodies will be able to form a complex that can be detected. Monovalent antibodies present in the sample will only be able to bind the capture agent bound to a solid support or the detection agent present in the liquid phase. Monovalent antibodies and other materials that did not bind to the capture agent on the solid support can then be removed from the solid support during a wash step. These molecules that are bound to capture agent but not detection agent will not be detected by the assay, allowing a specific quantitation of bivalent molecules.

For the capture step, a solid support is coated with an capture agent. A capture agent used in the capture step may be directly or indirectly linked to the solid support. For direct linkage to a solid support, such as an ELISA-based assay format using a microtiter plate, the capture agent may be diluted in a buffered solution, such as phosphate buffered saline and spread on each well of an assay plate. For indirect linkage, the capture agent can be immobilized to the solid support through a secondary antibody or binding reagent such as biotin-avidin system. For example, avidin or streptavidin may be immobilized on a microtiter plate, and a capture agent is labeled with biotin to facilitate linkage of the capture agent to the avidin or streptavidin coated plate.

Alternatively, capture agent may be linked directly to a membrane. Membranes may be spotted with capture agent using, e.g., a slot or dot blot apparatus. The slot or dot blot assay of the present invention is similar in principle to the microtiter plate assay, with the exception that a membrane is substituted for the plastic microtiter plate, and capture agents are applied to the membrane as a slot or dot.

Biotin conjugation of antibodies is well-known in the art and biotin is typically conjugated to proteins through primary amines (i.e., lysines). Biotin may be obtained from commercial sources such as Pierce EZ link Sulfo-NHS-LC biotin or Pierce NHS-LC biotin II and conjugated to an antibody according to the manufacturer's instructions. Additionally, kits for biotin conjugation may be obtained from such companies as Sigma Aldrich, Alpha Diagnostic International, and Amersham Pharmacia Biotech.

A detection agent is used to detect the desired antibody in the assay. In certain embodiments, the detection agent may be the same as the capture agent. In other embodiments, the detection agent may recognize a different region of the desired antibody than the capture agent, such as a different epitope.

For the detection step, the detection agent is labeled with a detectable compound or composition. In exemplary embodiments, the label is an enzyme that catalyzes a chemical reaction in the presence of a substrate compound. Exemplary substrate compounds will generate a chromogenic reaction product that may be detected by spectrophotometric, fluorimetric, or by visual means. In an exemplary embodiment, spectrophotometric detection is used when the assay described herein is conducted using a microtiter plate. Detection may also be accomplished by a visual comparison to with a set of similarly prepared standards. Other exemplary substrate compounds include substrate compounds that utilize chemiluminescent moieties that generate a light reaction.

In an exemplary embodiment, the detection agent of the present invention is labeled with horseradish peroxidase (HRP) enzyme. Methods to label antibodies with HRP are well-known in the art. HRP-antibody conjugates may be prepared using activated peroxidase according to the manufacturer's instructions. HRP conjugation kits for labeling antibodies may be obtained from, e.g., Alpha Diagnostic International or Zymed Laboratories. Chromogenic substrates that are reactive with horseradish peroxidase enzyme include, but are not limited to, 3,3′,5,5′ tetramethylbenzidine (TMB), 2,2-azino-di-(3-ethylbenzthiazoline sulfonic acid) (ABTS), o-phenylenediamine dihydrochloride (OPD).

In another exemplary embodiment, the detection agent of the present invention may be labeled with alkaline phosphatase. Methods to label antibodies with alkaline phosphatase are well-known in the art. Alkaline phosphatase-antibody conjugates may be prepared using activated alkaline phosphatase according to the manufacturer's instructions. Alkaline phosphatase conjugation kits for labeling antibodies are available from Zymed Laboratories, Merck or Roche. Substrates for Alkaline phosphatase (AP) include, but are not limited to, 5-bromo, 4-chloro, 3-indolyphosphate (BCIP substrate), 5-bromo, 4-chloro, 3-indolyl phosphate/nitroblue tetrazolium/iodonitrotetrazolium (BCIP/INT substrate), 5-bromo, 4-chloro, 3-indolyphosphate/nitroblue tetrazolium (BCIP/NBT substrate). In an exemplary embodiment, using an ELISA-based assay format, p-Nitrophenyl phosphatase (PNPP) may be the preferred chromogenic substrate.

Enzyme-labeled detection agent can also react with chemiluminescent moieties to generate light. Examples of particularly useful chemiluminescent labeling compounds are luminol, isoluminol, theromatic acridinium ester, imidazole, acridinium salt, and oxalate ester. Likewise, a bioluminescent compound can be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin. In an exemplary embodiment, horseradish peroxidase enzyme reacts with a chemiluminescent compound such as luminol to generate light. Emitted light can be measured in an ELISA based assay format or in an immunoblot based assay format using methods that are well-known in the art.

Detection may also be accomplished using a variety of other approaches. For example, the detection agent may be radioactively labeled. The radioactive isotope (e.g., ¹²⁵I, ¹³¹I, ³⁵S or ³H) may be detected by such means as the use of a gamma counter, a scintillation counter or by autoradiography.

It is also possible to label the detection agent with a fluorescent compound. When the fluorescently labeled detection agent is exposed to light of the proper wavelength, its presence can then be detected. Commonly used fluorescent labeling compounds that may be used are presented above.

Labeled detection agent may be added to a solid support coated with capture agent followed by a reference standard or a sample to be quantitated. Alternatively, a reference standard or a sample to be quantitated may be added to a solid support followed by the addition of labeled detection agent. Sample may be incubated with labeled detection agent as described herein under appropriate conditions and for a sufficient period of time.

After incubation, the solid support is washed to remove unbound substances. Exemplary wash solutions are well-known in the art and may include phosphate buffered saline and Tris-buffered saline. The wash step may be repeated 1, 2, 3 or more times. In exemplary embodiments, a substrate reactive with the detectable label will be added following the wash and a stop solution such as 0.2 N H₂SO₄ may be added before a color change measured.

A colored reaction product may be read using a spectrophotometer or an ELISA plate reader. The amount of color reaction product generated is proportional to the amount of test antibody molecules in the bivalent form. If the detection agent is labeled with horseradish peroxidase enzyme (HRP) and the chromogenic substrate is TMB, a blue reaction product will be generated and may be measured by the absorbance of light, preferably measured after stopping the reaction by addition of an acidic solution such as 0.2 N H₂SO₄, which change the color from blue to yellow. If the detection agent is labeled with HRP and the chromogenic substrate is ABTS, a green reaction product will be generated and optical density may be measured. Further, when the antibody is labeled with HRP and the chromogenic substrate is OPD a yellow-orange reaction product will be generated and optical density may be measured. If the detection antibody is labeled with alkaline phophatase and the chromogenic substrate is PNPP, a yellow reaction product will be generated and optical density may be measured.

Quantification may be performed by a comparison of absorbance readings of the samples to a standard curve. A standard curve may be created by measuring known amounts of test antibody (i.e., reference standards) in the assay and measuring the absorbance of each test concentration. In an exemplary embodiment, at least about 5, 6, 7, or 8 concentrations of test antibody will be measured to generate a standard curve. The amount of test antibody in the sample may be measured using a relationship between concentrations versus absorbance of the standards.

If high levels of test antibody are expected or present in the sample, the sample may be diluted prior to the assay. For sample requiring dilution, the linearity and accuracy of dilution should be determined to ensure that the results will be reliable after dilution. Linearity of dilution refers to the ability of the analytical method, within the assay range to obtain test results that are close to the expected concentration of the analyte in the diluted sample. Linearity is measured by the r-squared (r² coefficient of determination, or r, coefficient of correlation), value for the linear regression of the expected versus observed concentration while accuracy is measured by the percent recovery.

These labeling and detection methods serve as examples of the variety of compounds and measuring methods available and are not intended to be limiting. A skilled artisan would readily recognize other methods or compounds useful in the invention.

### Kits

Also provided herein are kits for detecting test antibody in a sample. Kits may comprise one or more reagents for detecting a test antibody in a sample, including an anti-idiotype antibody that is immunospecific for the test antibody. The anti-idiotype antibody may be provided as an unlabeled and/or labeled antibody. If a labeled detection antibody is not provided, the antibody itself may be labeled with a detectable marker, e.g., a chemiluminescent, enzymatic, fluorescent, or radioactive moiety. If a labeled detection antibody is provided, an appropriate chromogenic substrate may be provided.

In certain embodiments, a kit for detecting a test antibody in a sample may further comprise a reference standard, wherein the reference standard is purified test antibody that may be spiked into a sample. The kit may additionally comprise a solid support such as a reagent strip that contains immobilized capture anti-idiotypic antibody. Alternatively, the kit may comprise a sterile microtiter plate or strip upon which the capture anti-idiotypic antibody may be immobilized. In further embodiments, a kit for detecting a test antibody in a sample may still further comprise instructions for detecting the level of a bivalent test antibody in a sample. Reagents in the kit may be provided in individual containers or as mixtures of two or more reagents in a single container.

### Exemplification

The invention, having been generally described, may be more readily understood by reference to the following examples, which are included merely for purposes of illustration of certain aspects and embodiments of the present invention, and are not intended to limit the invention in any way.

### EXAMPLE 1

### Quantitative Determination of a Bivalent Antibody anti-CD4 in Human Serum using a Bridging-based Enzyme-Linked Immunosorbet Assay

A. Coating of Assay Plates with Anti-Idiotypic-Antibody

Plates were coated with 10 μg/ml anti-Id-anti-CD4 monoclonal antibody. Briefly, the antibody was diluted to 10 μg/ml in phosphate buffered saline to make the coating solution. 200 μl of coating solution was added to each well of a Costar 96-well plate and incubated for 16-24 hrs in a humidified chamber.

The plates were aspirated and excess solution was removed by slapping the plates on a paper towel. 200 μl of blocking solution (10 mM phosphate buffer, pH 7.4 containing 0.5% ProClin-300 (Supelco), 1% BSA, and 2.5% sucrose) was added immediately to each well and the plates were incubated overnight in a humidified chamber. The plates were then aspirated and excess solution removed before drying at room temperature. Dried plates were sealed in plastic bags and stored at 2-8° C.

HRP-conjugated anti-CD4 antibody was prepared using activated peroxidase according to the manufacturer's instructions (Zymed Laboratories). A conjugate stock at 100 μg/ml was made in 50% glycerol/PBS with 1% bovine serum albumin (BSA) and stored at −20° C. A working dilution was prepared by further diluting the stock solution in the same buffer to make a 50× solution. The final concentration used in the assay was 143 ng/ml anti-CD4-HRP (at 1:700 dilution of the original stock in 50 mM Tris buffer, pH 7.4, containing 1% BSA, 0.05% TWEEN™ 20, and 0.5% ProClin-300 (ProClin-300 is a broad spectrum anti-microbial commercially available from Zymed, which acts as a preservative for enzymes)).

B. Assay Procedure

Two microtiter plates were used for a single assay run. One plate was an uncoated low binding round bottom plate and the other was an anti-Id-anti-CD4 coated plate. Samples were added to the uncoated plate first and then were transferred to the anti-Id-anti-CD4 coated plate to eliminate any time-shift effect by reducing the time needed to add the samples to the wells. 10 μl reference, control or sample was added per well to an uncoated 96-well plate. Three wells were used per reference and sample. 60 μl of assay buffer (50 mM Tris buffer, pH 7.4, containing 1% BSA, 0.05% TWEEN™ 20, and 0.5% ProClin-300) was pipetted into each well of the uncoated plate containing the reference or samples, using a multi-channel pipette. A dilution of anti-Id-anti-CD4-HRP conjugate (6 ml per plate) was made in the assay buffer described above and poured into a reservoir. The dilution factor was based on the conjugate lot at a 1:700 dilution or 143 ng/ml. 50 μl of the diluted conjugate was pipetted into each well of an anti-Id-anti-CD4 (10 μg/ml) coated plate using a multi-channel pipette. A 50 μl aliquot mixture of sample and assay buffer from each well of the uncoated plate was transferred to an anti-Id-anti-CD4 coated plate containing 50 μl of anti-Id-anti-CD4 conjugate per well using a multi-channel pipette. Assay plates were incubated at room temperature on a shaker at 700 rpm for 90±5 min. Assay plates were washed in 50 mM Tris buffer, pH 7.4 containing 0.9% NaCl, 0.05% TWEEN™ 20 and 0.5% ProClin-300. Each well was washed 4 times. 100 μl of TMB was pipetted into each well using a multi-channel pipette and each plate was incubated at room temperature on a shaker at 700 rpm for 5 to 6 min. 100 μl of stop solution (0.2 N H₂SO₄) was added to each well and the plate was placed briefly on the plate shaker. Assay plates were read at 450 nm using 590 nm as the reference filter, using a program set up in REVALATION® software in Dynex plate reader, which fits a calibration curve using linear regression on log transformed concentration and log-transformed OD. All of the plates were read and the data processed using the REVALATION® program immediately after the plate was read.

Percent relative error in curve fitting at each reference was calculated using the built-in function in Dynex REVALATION® Software as:

[(Nominal concentration−back calculated concentration)×100] Nominal concentration

The data from 12 runs were compiled to demonstrate the adequacy of the model fitting. Mean error at each reference level and mean error for each run was less than 10%. Further, for each run, at least 6 out of 8 non-zero references had less than 10% error.

In the assay protocol described above, a reaction does not occur between the anti-CD4 coated on the plate and the HRP conjugated anti-CD4. However, to determine if a time-shift effect occurred between the time the sample was added to the first row of the assay plate and the last row of the assay plate, we conducted a study wherein CD4-HRP and anti-ID-anti-CD4 were added to a set of controls at time zero and then to additional control sets at time 1, 2, and 3 minutes. No time-shift effect was observed up to 3 minutes following the calibration curve. Thus, addition of CD4-HRP and anti-Id-anti-CD4 can take up to 4 minutes without any significant effect in the assay. CV % among the four test sets are less than 10% indicating no adverse effect of time shifting CV, as used herein, refers to the coefficient of variation, which is a quantitative measure of precision that is expressed relative to the observed or theoretical (nominal) mean value.

The analytical sensitivity or limit of detection (LOD) of the assay was calculated using the average reading of zero standard (Reference A), the next higher standard (Reference B at 25 ng/ml, see equation) and the standard deviation of zero standard from 10 replicates in a single assay using the following formula:

$$LOD = \frac{[ ( B - A ) ( ng / ml ) \times 2 SD of OD of Ref A ]}{( OD of Ref B - OD of Ref A )} .$$

By this method, the limit of detection, which is the lowest concentration of anti-CD4 that this assay can reliably differentiate from background noise, is 3.5 ng/ml (mean of 2 such determinations). The upper confidence limit of sensitivity for the assay is 8.7 ng/ml

The lowest limit of quantitation (LLOQ) and upper limit of quantitation (ULOQ) were established by determining the mean and CV % among 5 replicates of a samples containing the analyte at 25 ng/ml and 2500 ng/ml, respectively. The LLOQ is defined as the lowest concentration of the analyte the assay can measure with less than or equal to 20% relative error and less than or equal to 20% CV, whereas the ULOQ is defined as the highest analyte concentration that can be measured with less than or equal to 15% relative error and less than or equal to 15% CV.

To determine the LLOQ and ULOQ each assay was performed in duplicate. The mean value of the sample containing 25 ng/ml should be ≦30 ng/ml (mean+20%) and the mean value of the sample containing 2500 ng/ml should be ≧2125 ng/ml (mean−15%). Samples containing anti-CD4 at 25 ng/ml were quantitated by the assay within 20% bias and CV and samples containing 2500 ng/ml anti-CD4 were quantitated by the assay within 15% bias and CV. Thus, the assay range is defined as 25-2500 ng/ml.

Further, we determined the intra-assay precision of the assay, which is a measurement of the closeness of the agreement between a series of measurements obtained from multiple sampling of the same homogenous sample within a single assay. Twenty replicates of each control were run in a single assay and the mean, standard deviation, and CV % were determined for each control. The CV % for controls containing 75 ng/ml, 600 ng/ml, and 1800 ng/ml anti-CD4 were 4, 5, and 5%, respectively.

We also determined the inter-assay precision of the assay, which expresses the closeness of agreement between a series of measurements obtained from multiple sampling of the same homogeneous sample between multiple assays. In order to determine the reproducibility of the assay described herein, the inter-assay precision study was performed by running the three controls containing anti-CD4 in different runs on different days. The mean, standard deviation and CV % of the three controls were calculated. The inter-assay coefficients of variation (CV) for the control samples at 75 ng/ml, 600 ng/ml, and 1800 ng/ml were 9, 7, and 5%, respectively. Mean % bias was calculated as (1−Mean/Target)*100 and the total error was calculated as [% CV+% Bias].

Acceptance ranges for three controls at low, medium and high anti-CD4 levels were determined from the concentration values from each replicate from these assays using mean±3*SD

Interference was also studied together with the linearity of dilution, since monovalent anti-CD4 can bind to the assay plate coated with anti-CD4, which may result in non-linearity of dilution and an under-estimated bivalent anti-CD4 measurement. Other possible sources of interference could result from bispecific anti-CD4, anti-CD4 degradation products containing target epitopes, free anti-CD4, a complex of drug plus anti-drug antibodies with a single free epitope on the drug, a complex of drug plus anti-drug antibodies with a single free epitope on the anti-drug antibody, a complex of drug plus anti-drug antibodies with a multiple free epitopes on the drug, a complex of drug plus anti-drug antibodies with a multiple free epitopes on the anti-drug antibody. Negative interference is possible in each case except for a complex of drug plus anti-drug antibodies with a multiple free epitopes on the drug.

To determine the accuracy, spiking recovery experiments were carried out. Patient serum samples were spiked at 300, 600 and 900 ng/ml anti-CD4 using a 20× stock. This gave a spiking volume of 5%. Samples were then assayed and the percent recovery was determined by dividing the observed value by the expected concentration.

$$\% Recovery = \frac{Observed Concentration after Spiking \times 100}{Expected Concentration}$$

The Expected Concentration is equal to the spiked concentration plus background. Recovery in spiked patient pre-dose samples ranged from 77-124%. Additionally, 32 normal individual human serum samples were spiked in the range of 150-2000 ng/ml anti-CD4 and recovery in these samples ranged from 80-110%.

Further, linearity of dilution was tested to determine the reliability of measurements following dilution, since high serum levels of anti-CD4 are expected in patients treated with anti-CD4 and serum will likely need to be diluted prior to the assay. Dilution linearity refers to the ability of the analytical method, within the assay range, to obtain test results that are close to the expected concentration of the analyte in the diluted sample. Linearity is measured by the r² for the linear regression of expected vs. observed concentration while the accuracy is measured by the percent recovery.

Six samples were prepared by spiking anti-CD4 into three individual normal serum and three patient pre-dose samples at 2500 ng/ml and then diluted serially 1:2, 1:4, 1:8 and 1:16 in pooled normal human serum (equivalent to zero standard). The diluted samples were tested in the assay and a percent recovery was calculated for each dilution. The r² values for the three individual normal serum samples were 0.9957, 0.9948, and 0.9966, respectively. The r² values for the three patient pre-dose sample were 0.9987, 0.9987, and 0.9962, respectively.

As indicated above, monovalent anti-CD4 present in the sample may bind to either capture or detection antibody in the assay, and thus, may interfere with the measurement of bivalent anti-CD4. Interference, however, may be identified by non-linearity of dilution and may be apparent by the over recovery at higher dilutions. Using post-dose patient samples, which may contain monovalent anti-CD4, we investigated interference together with dilution linearity.

Three patient post-dose serum samples containing endogenous anti-CD4 were obtained after 1 hour, 12 hours, and 2 days. Initial dilution factor were determined to be 1:1.5 for the 1 hour sample, 1:10 for the 12 hour sample, and 1:2 for the Day 2 sample. These dilution factors were determined according to the concentration previously measured in a anti-CD4 competition assay. All samples were further serially diluted to 1:2, 1:4, 1:8, and 1:16 and tested. The sample collected 1 hour after treatment was linear in the dilution, recovering 87-106% at all the dilutions. For the 12 hour sample, only dilutions above 1:20 were linear and for the Day 2 sample, only dilutions above 1:8 were linear. These results suggest that an interfering substance may be present at higher concentrations, but that at and above certain dilutions the effect of the interfering material can be eliminated.

For further study, two additional patients were selected and all samples which had anti-CD4 readings in previous competition assay were analyzed in the assay. From one patient, samples were obtained after 1, 6, 12, and 24 hour, and from the second patient, samples were obtained after 30 min, 1, 7, 14, 21 and 28 days.

Recovery was calculated using two methods. One method used the observed value at lowest dilution and the other method used the observed value at the highest dilution. In the conventional method, the expected value was calculated by dividing the observed value at lowest dilution by the dilution factor. In the second method, the expected value was calculated by multiplying the observed value at highest dilution by the dilution factor. When no interference is present, either method would give 100% recovery. When interference is present, but eliminated by dilution, recovery will be Back calculated sample values were obtained as equal to the assay value multiplied by the dilution factor, and each sample value is presented as a percent of the sample value calculated at the next higher and lower dilution. As expected, the samples at lower dilution showed non-linearity and as they were diluted further, they reached the linearity and recovered within 100±10%.

Samples containing high concentrations of anti-CD4 were also measured. Pooled human serum samples spiked with anti-CD4 up to 1 mg/ml were tested in the assay and OD was measured. Anti-CD4 concentrations above 5000 ng/ml had a lower OD than samples with a concentration of 2500 ng/ml indicating a hooking effect. Thus, to ensure optimal accuracy all samples should ideally be tested at minimum of two dilution levels.

Sample stability was determined by testing the anti-CD4 in human serum subjected to several different storage conditions likely to be encountered during clinical sample analysis. For example, aliquots of three serum samples from one study containing low, medium and high concentration of anti-CD4 were subjected to following short term storage conditions: 1) storage at −70° C. freezer until all samples are ready for analysis; 2) storage at room temperature for 4 hours, then at −70° C. until analysis; 3) storage at 2-8° C. for least 24 hours, then at −70° C. until analysis; or 4) storage under freeze-thaw cycles between −70° C. and room temperature. To investigate storage conditions under freeze-thaw cycles, 2 sets of 3 samples were prepared as follows: freeze both sets at −70° C. for at least 4 hours, and then thaw them at room temperature for 2 hours (first cycle). Repeat this freeze/thaw cycle 2 more times and freeze back one set until ready for analysis. Continue freeze-thaw cycling of the second set for another 3 times and freeze back. On the day of the assay, when the two sets of samples are thawed, one has gone through 4 cycles and the other 8.

To determine sample stability, all samples were tested in replicates of 5 and the stability was determined by the percent recovery, which is equal to the observed value divided by the expected value multiplied by 100, where the expected value is the concentration of the sample aliquots stored at a −70° C. freezer.

Anti-CD4 recovery after 3 storage and stress conditions ranged from 95% to 106% indicating that the samples have acceptable stability. All samples showed CV % between 2-10%. The data indicated that that samples could be frozen and thawed at least 8 times without having any adverse effect on bivalent anti-CD4 concentrations.

Method Correlation between anti-CD4 Competition EIA (Enzyme Immunoassay) and Bivalent Assays was done using samples from individual normal human serum spiked with anti-CD4 over the range of 150-2500. Thirty such samples were run in Competition EIA as well as Bivalent anti-CD4 assay in two formats; one using the method described above and the other using CD4-HRP as the detection antibody.

Initial testing was done using a reference lot, which was prepared in lipid-stripped serum. The data was plotted as anti-CD4 concentration as determined by competition assay vs. bivalent assay. Slopes of the curves were 1.5 and 1.2 for the two bivalent formats. Least square regression analyses were performed to determine if the observed intercept and slope were statistically significantly different from 0 and 1, respectively. In both formats, the slope values were significantly above 1 indicating the bivalent assay values were significantly higher than competition values. The r² value was 0.972 for the bivalent assay with CDR-HRP and 0.9519 for the bivalent assay with anti-Id-anti-CD4.

Thirty-two similarly prepared samples were then tested using a reference lot prepared in non-lipid stripped (unprocessed) pooled normal human serum. Data were then analyzed in a similar fashion. The analysis revealed that, when samples were analyzed using the reference prepared in unprocessed serum the assay values from Competition EIA match well with Bivalent anti-CD4 assays in both formats. In both cases there were non significant intercept, indicating the regression line passing through zero, and slope was not significantly different from 1 (p<0.005). The r² values were 0.9246 for the bivalent assay with CDR-HRP and 0.939 for the bivalent assay with anti-Id-anti-CD4.

Thus, as indicated by the above results, plates coated with anti-idiotypic-anti-CD4 on the solid phase for capture and HRP-conjugated anti-idiotypic-anti-CD4 for detection provide a reliable ELISA for the determination of bivalent anti-CD4 in human serum. The assay for anti-CD4 has a dynamic range (LLOQ-ULOQ) of 25-2500 ng/ml with a minimum detection limit of 3.5 ng/ml. Further the specificity study showed no interference by human IgG, but due to the presence of cross-reactivity and interference by hemoglobin, hemolyzed samples should be avoided. Finally, to minimize interference by the presence of monovalent anti-CD4 molecules, each sample should be diluted before testing and to assure that the dilution is sufficient to eliminate the interference, samples should be tested in at least two dilutions and confirmed the two values back calculated with dilution factor gives a value within 100±15% of each other. If a larger dilution recovers more than 15% compared to the lower dilution, the sample should be diluted further and tested in the assay as indicated from the data on the method comparison.

### EXAMPLE 2

### Quantitative Determination of Bivalent Anti-CD4 in Human Serum Using an Avidin-Biotin based Enzyme-Linked Immunosorbent Assay

A. Coating of Assay Plates with Avidin

Assay plates, such as microtiter plates were coated with avidin. Briefly, avidin was diluted to 7.5 μg/ml in phosphate buffered saline (PBS) to make the coating solution. 200 μl of coating solution was added to each well of a 96-well plate (Costar) and incubated for 3 days in a humidified chamber. The plates were aspirated and excess solution was removed by slapping the plated on a paper towel. 200 μl of blocking solution made in PBS (10 mM phosphate buffer, pH 7.4) containing 0.5% ProClin-300 (Supelco), 1% BSA, and 2.5% sucrose) was added immediately to each well and the plates were incubated overnight in a humidified chamber. The plates were then aspirated and the excess solution removed before drying at room temperature. Dried plates were sealed in plastic bags and stored at 2-8° C.

B. Assay Procedure

Using avidin coated plates, 20 μl of anti-CD4 reference or sample was transferred into each well. 50 μl of anti-id-anti-CD4-HRP conjugate and 50 μl of biotinylated anti-id-anti-CD4 was added to each well. The plates were incubated at room temperature on a plate shaker (˜700 rpm) for 60 minutes and then washed four times using a Bio-Rad Immunowash. 100 μl of TMB substrate solution was added to each well and the plates were incubated at room temperature on a plate shaker (˜700 rpm) for 10 minutes. 100 μl of Stop Solution (0.2 N H₂SO₄) was added to each well and absorbance at 450 nm with 590 nm as a reference was measured on a plate reader (MRX Revelation, Dynex Technologies).

Reference standards were made in human serum by adding known amount of anti-CD4. The concentrations of anti-CD4 were 0, 30, 100, 300, 500, 1000, 2000 and 3000 ng/ml. The highest reference was limited to 3000 ng/ml as the assay showed a hooking effect beyond 5000 ng/ml.

All plates were read using the Dynex MRX plate reader. A standard curve was obtained for each assay using linear fit with Log/Log scale. Data were processed using the Revelation program immediate after the plate was read.

C. Experimental Results

The reproducibility of the standard curve was examined using the data from 12 assays. The CV % of the intercept and slope for the 12 assay runs was 14 and 12%, respectively. The mean r²-squared (r²) value across the assay runs was 0.995.

The lower limit of detection (LOD) of the assay was calculated using the average reading of zero standard, the 100 ng/ml standard, and the standard deviation of zero standard from 10 replicates in a single assay. By this method, the LOD, which is the lowest concentration of anti-CD4 that this assay can reliably differentiate from background noise, is 16.5 ng/ml (mean of 2 such determinations).

The upper limit of quantitation (ULOQ) and lower limit of quantitation (LLOQ) were also determined. For these calculations three replicates of standards were run as unknowns with the reference curve and assay values were assigned. Mean, SD, and 95% lower and upper confidence interval (LCI and UCI were calculated as (Mean−1.96*SD) and (Mean+1.96*SD) respectively) were calculated. Bias or % Relative Error of mean, LCI and UCI was calculated as:

(Observed)−1)*100/(Nominal)

In each case, the percent relative error was plotted against nominal or target value and a precision profile was determined by plotting CV % versus concentration. The estimates of LLOQ and ULOQ were obtained as 70 ng/ml and 3000 ng/ml respectively. Therefore, the assay dynamic range is between 70 and 3000 ng/ml.

Precision of the assay was measured both as intra- and inter-assay precision. To measure intra-assay precision, twenty replicates of each control were run in a single assay and the mean, standard deviation, and CV % were obtained for each control. The CV % for anti-CD4 controls of 207 ng/ml (low), 842 ng/ml (medium), and 1814 ng/ml (high) were 13.8%, 9.4%, and 6.8%, respectively.

In order to determine the reproducibility of this assay, an inter-assay precision study was performed by running the same three controls containing anti-CD4 in different runs in different days. The means, standard deviations and CV % of the three controls were calculated. The inter-assay coefficients of variation for the control samples, once again at low, medium and high concentrations were 14.7%, 9.5% and 7.7%, respectively. To determine the specificity of the assay, anti-CD4 was measured in samples containing other components that are structurally similar to anti-CD4. In these studies, known amounts of human IgG, humanized IgG and hemoglobin were added to human serum spiked with different concentration of anti-CD4. Only hemoglobin showed significant cross-reactivity and interference in the anti-CD4 assay. Thus, hemolyzed samples or samples contaminated with hemoglobin should be avoided. Human IgG and humanized IgG did not appear to cross-react or interfere with anti-CD4 in the assay.

Linearity and accuracy of the dilution were also investigated since high levels of anti-CD4 are expected in human serum. Four samples were prepared by spiking anti-CD4 into individual normal serum at 2400 ng/ml and then diluted serially 1:2, 1:4, 1:8 and 1:16 in pooled normal human serum (zero standard). The diluted samples were tested in the assay and a percent recovery was calculated for each dilution. Undiluted samples recovered 63-77% indicating some interfering substance in the samples. The anti-CD4 amount in the lowest dilution tested may be at or close to the lower limit of quantitation of the assay and therefore not reliable. Dilutions at 1:2 to 1:8 shows mean recovery of 87%-107%. Matrix effect and dilution requirements were further investigated and are presented below.

In a further study, three individual human serum samples and a pooled normal human serum sample were spiked at 300, 600 and 900 ng/ml anti-CD4 using 20× stock so the spiking volume was 5%. Samples were then assayed and the percent recovery was determined by dividing the observed value by the expected value. An unspiked sample value was assumed to be zero for the calculation. The mean recovery range was between 93-118% (mean±SD is 107±13%) for the three samples and the pooled serum sample.

To determine the consequences of the time difference between the addition of the first sample and the addition of the last sample, three sets of controls (low, medium, and high) in triplicates were added to the plate in 10, 15 and 20 minutes after a standard curve and the first set of controls (time zero) were added. The samples were then assayed. The means of the 3 replicates were 143.9, 660.8, and 2940.2 ng/ml for the low, medium, and high controls, respectively. The CV % among the three sets are less than 10% indicating no adverse effect of time shifting.

High concentration were also tested using pooled human serum samples spiked with anti-CD4 at 20,000 and 10,000 ng/ml. Optical density (OD) readings were 0.08 and 0.34, respectively, indicating a hooking effect. Thus, all samples should to be tested at minimum of two dilution levels to ensure the quantitation is not affected by the hooking effect, by confirming that concentration of the more diluted sample is higher than the less diluted.

Further, a method correlation between anti-CD4 competition and bivalent assays was done using freshly made samples from both individual as well as pooled normal human serum samples spiked with anti-CD4 over the range of 200-2500 ng/ml. Data were analyzed by regression separately for the two groups. For individual samples, the correlation (R² value) was 0.938 and the 95% confidence interval (CI) for slope was 0.56-0.71, whereas the intercept was between 50-263. For pooled normal human serum samples, correlation (R² value) was 0.982 and the 95% CI for slope was 0.84-0.99. The intercept was not statistically significant (p=0.79) indicating a zero intercept or the regression line passing through zero.

The slope from normal individual sample groups, being significantly lower than 1 as well as lower than the pooled serum, indicated a possibility of some matrix effects in the former. It is possible that some biotin present in the individual serum at different levels interfered with the assay by blocking the binding sites on the plate, while the samples prepared in pooled normal serum contained a constant level of biotin similar to that in reference standards.

A second correlation study was done using 1:100 diluted individual samples spiked with anti-CD4. The data showed a correlation of 0.99 and a 95% CI of 0.86-0.94. The intercept was not statistically significant (p=0.79) indicating a zero intercept or the line passing through zero.

The effect of dilution factor was also studied to determine the minimum dilution needed to eliminate the matrix effect in the bivalent assay. Samples were prepared using individual serum samples from normal population as well as from anti-CD4 study population (pre-dose). These samples were diluted at different levels (1:10, 1:50 and 1:100) using pooled normal serum, which did not show any interference. The diluted samples, as well as undiluted, were then spiked with anti-CD4 stock solution to approximately 2000 ng/ml and tested in both competition and bivalent assays.

Undiluted normal samples in the bivalent assay were recovered at values 69-92% of the competition assay while samples at 1:10 and higher dilutions were recovered at values 88-119%. Similarly, undiluted pre-dose patient samples in the bivalent assay were recovered at 70-90% of the Competition assay while the samples at 1:10 and higher dilutions were recovered at 98-109%. This data indicates that a 1:10 dilution of a sample is sufficient to eliminate the matrix effect.

To determine the minimum dilution required for patient samples containing low level of anti-CD4, three pre-dose samples were diluted with pooled normal human serum at 1:2, 1:5 and 1:10. These dilutions as well as undiluted aliquots were spiked with anti-CD4 at low level (300 ng/ml). Samples were tested in both assays and recovery was calculated. Low range samples up to about 700 ng/ml showed mean ratio of bivalent values that were 107% that of the competition assay values. Further, undiluted samples can be used when the anti-CD4 level is lower than 500 ng/ml. Ideally, at low levels samples should be tested as undiluted and 1:2 diluted in pooled normal human serum.

Thus, as indicated by the above results, Avidin-coated assay plates and HRP conjugated and biotinylated anti-idiotypic antibody to anti-CD4 as conjugates provide a reliable ELISA-based assay for the determination of bivalent anti-CD4 in human serum. The assay has a dynamic range (LLOQ-ULOQ) of 30-3000 ng/ml with a minimum detection limit of 17 ng/ml. The assay shows intra- and inter-assay precision with coefficients of variation at low medium and high level of anti-CD4 of 13.8, 9.4 and 6.8% and 14.7, 9.5 and 7.7%, respectively. The specificity study showed no significant interference with human IgG. However, due to the presence of cross-reactivity and interference by hemoglobin, hemolyzed samples should be avoided. Based on the data from the method comparison and the linearity study samples should ideally be diluted before testing. However, when the anti-CD4 concentration is low (˜500 ng/ml) undiluted and 1:2 diluted samples can be tested. The shifting study suggests that samples can be added to the plate at least up to 20 minutes after addition of the reference standards.

### Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method for detecting a bivalent antibody comprising contacting a sample with capture anti-idiotypic antibodies and detection anti-idiotypic antibodies under appropriate conditions and for a sufficient period of time to allow binding; wherein detection of the detection anti-idiotype antibody indicates the presence of bivalent target antibodies in the sample.

2. The method of claim 1, wherein the bivalent antibody is polyclonal, monoclonal, chimeric, humanized or human.

3. The method of claim 1, wherein the bivalent test antibody is a tetramer, a F(ab′)₂ fragment, a diabody, a minibody, a scFv or a single domain antibody.

4. The method of claim 1, wherein the sample is a body fluid obtained from a mammal, such as blood, plasma, urine, interstitial fluid, lymph, gastric juices, bile, serum, saliva, sweat, spinal fluid or brain fluid.

5. The method of claim 1, wherein the capture anti-idiotypic antibody is linked to a solid support, wherein the capture anti-idiotypic antibody is directly linked or indirectly linked to the solid support.

6. The method of claim 5, wherein the solid support is a microtiter plate.

7. The method of claim 8, wherein avidin or streptavidin is on the solid support.

8. The method of claim 10, wherein the capture anti-idiotype antibody is labeled, such as by biotinylation.

9. The method of claim 1, wherein the detection anti-idiotypic antibody is the same as the capture anti-idiotypic antibody.

10. The method of claim 1, wherein the detection anti-idiotypic antibody is different than the capture anti-idiotypic antibody.

11. The method of claim 1, wherein the detection anti-idiotypic antibody includes a label, such as an enzyme, horseradish peroxidase or alkaline phosphatase.

12. The method of claim 11, wherein the detection anti-idiotypic antibody is detected by a spectrophotometer or plate reader.

13. A method of detecting a bivalent antibody in the presence of the bivalent antibody's monovalent form comprising: obtaining a sample containing both bivalent and monovalent forms of an antibody, contacting the sample with a capture anti-idiotypic antibody and a detection anti-idiotypic antibody, incubating said sample for sufficient time to allow binding of the antibodies, treating the sample to remove unbound antibody and measuring for the presence of detection antibody, wherein presence of detection idiotype antibody indicates the presence of bivalent antibodies in the sample.

14. A kit for detecting a bivalent test antibody in a sample, comprising a capture anti-idiotypic antibody against the test antibody and a detection anti-idiotypic antibody against the test antibody.

15. The kit of claim 13, further comprising a solid support, such as a microtiter plate, or a nylon or cellulose membrane.

16. The kit of claim 14, wherein the capture anti-idiotypic antibody is directly or indirectly linked to the solid support.

17. The kit of claim 13, wherein the detection anti-idiotypic antibody is labeled, such as with horseradish peroxidase enzyme or alkaline phosphatase.

18. The kit of claim 13, further comprising a chromogenic substrate.
